(19) United States
(12) Patent Application Publication
(10) Pub. No.: US 2026/0289294 A1
(43) Pub. Date: Sep. 24, 2026

(54) METHOD AND DEVICE FOR TRAINING EMOTIONAL SITUATION RECOGNITION MODEL

(71) Applicant: ELECTRONICS AND TELECOMMUNICATIONS RESEARCH INSTITUTE, Daejeon (KR)

(72) Inventors: Kyoung Ju NOH, Daejeon (KR); Se Won Oh, Daejeon (KR); Hyun Tae Jeong, Daejeon (KR)

(21) Appl. No.: 19/330,008

(22) Filed: Sep. 16, 2025

(30) Foreign Application Priority Data
Mar. 19, 2025 (KR) 10-2025-0035088

(51) Int. Cl. G06N 3/08

(52) U.S. Cl. CPC G06N 3/08

(57) ABSTRACT

A method and device for training an emotional situation recognition model are provided. The method includes obtaining profile information of a user, lifelog data of the user, and emotion journal data written about a first emotion caused by an event, extracting context information about the first emotion based on the emotion journal data, segmenting only lifelog data corresponding to a time interval in which the event occurred from each piece of the lifelog data, based on the context information, and training the emotional situation recognition model for recognizing a second emotion of the user, based on the profile information, the segmented lifelog data, and the context information.

### CROSS-REFERENCE TO RELATED APPLICATION

This application claims the benefit of Korean Patent Application No. 10-2025-0035088, filed on March 19, 2025, in the Korean Intellectual Property Office, the entire disclosure of which is incorporated herein by reference for all purposes.

### BACKGROUND

### Field of the Invention

One or more embodiments relate to a method and device for training an emotional situation recognition model.

### Description of the Related Art

With recent development of services based on user experience data, various products utilizing life-logging technology are being commercialized. Users may record their location information, activity information, and/or emotions through applications installed on their smartphones for health management, safety management, or social connection. The recorded lifelog data may be analyzed for health care of the users. The lifelog data may also be utilized in content recommendation services or for information sharing among users.

As the services based on user experience data expand, demand for affective computing services to improve emotional quality of life of individuals is also increasing. For example, the affective computing services may provide customized psychological stabilization functions based on user experience data to alleviate psychological stress or negative emotions.

The above description has been possessed or obtained by the inventor(s) in the course of conceiving the present disclosure and is not necessarily an art publicly known before the present application is filed.

### SUMMARY

Embodiments provide a method of training an emotional situation recognition model based on profile information of a user, segmented lifelog data, and context information.

Embodiments provide obtaining a model for accurately predicting emotions of a user.

However, the technical aspects are not limited to the aforementioned aspects, and other technical aspects may be present.

According to an aspect, there is provided an emotional situation recognition model including obtaining profile information of a user, lifelog data of the user, and emotion journal data written about a first emotion caused by an event, extracting context information about the first emotion based on the emotion journal data, segmenting only lifelog data corresponding to a time interval in which the event occurred from each piece of the lifelog data, based on the context information, and training the emotional situation recognition model for recognizing a second emotion of the user, based on the profile information, the segmented lifelog data, and the context information.

The emotion journal data may include an utterance of the user and a text input by the user.

The context information may include the event, the time interval, the first emotion, and location information in which the event occurred.

The lifelog data may include a plurality of types of lifelog data, and the plurality of types of lifelog data may include sound data of surroundings of the user, biometric data of the user, and activity data of the user.

The training may include labeling each piece of the segmented lifelog data with the first emotion.

The training may include recognizing the second emotion based on the lifelog data labeled with the first emotion.

The training may include training the emotional situation recognition model based on the first emotion and the second emotion.

The extracting may include inputting the emotion journal data to a large language model (LLM) to extract the context information about the first emotion.

The profile information may include gender, age, and personality of the user.

According to another aspect, there is provided an electronic device including a memory including instructions and at least one processor electrically connected to the memory and configured to execute the instructions, wherein the instructions, when executed by the at least one processor, may cause the electronic device to obtain profile information of a user, lifelog data of the user, and emotion journal data written about a first emotion caused by an event, extract context information about the first emotion based on the emotion journal data, segment only lifelog data corresponding to a time interval in which the event occurred from each piece of the lifelog data, based on the context information, and train an emotional situation recognition model for recognizing a second emotion of the user, based on the profile information, the segmented lifelog data, and the context information.

The emotion journal data may include an utterance of the user and a text input by the user.

The context information may include the event, the time interval, the first emotion, and location information in which the event occurred.

The lifelog data may include sound data of surroundings of the user, biometric data of the user, and activity data of the user.

The instructions, when executed by the at least one processor, may cause the electronic device to label each piece of the segmented lifelog data with the first emotion.

The instructions, when executed by the at least one processor, may cause the electronic device to recognize the second emotion based on the lifelog data labeled with the first emotion.

The instructions, when executed by the at least one processor, may cause the electronic device to train the emotional situation recognition model based on the first emotion and the second emotion.

The instructions, when executed by the at least one processor, may cause the electronic device to input the emotion journal data to a large language model (LLM) to extract the context information about the first emotion.

The profile information may include gender, age, and personality of the user.

Additional aspects of embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a schematic diagram illustrating a system of training an emotional situation recognition model, according to an embodiment;

FIG. 2 is a diagram illustrating a training device disclosed in FIG. 1;

FIG. 3 is a diagram illustrating a method of obtaining context information, according to an embodiment;

FIG. 4 is a diagram illustrating an operation of segmenting lifelog data, according to an embodiment;

FIG. 5 is a diagram illustrating a method of training an emotional situation recognition model, according to an embodiment;

FIG. 6 is a diagram illustrating a method of obtaining lifelog data and emotion journal data, according to an embodiment;

FIG. 7 is a flowchart illustrating a method of training an emotional situation recognition model, according to an embodiment; and

FIG. 8 is a schematic block diagram of a training device according to an embodiment.

### DETAILED DESCRIPTION

The following detailed structural or functional description is provided as an example only and various alterations and modifications may be made to the embodiments. Accordingly, the embodiments are not construed as limited to the disclosure and should be understood to include all changes, equivalents, and replacements within the idea and the technical scope of the disclosure.

Although terms of "first," "second," and the like are used to explain various components, the components are not limited to such terms. These terms are used only to distinguish one component from another component. For example, a first component may be referred to as a second component, or similarly, the second component may be referred to as the first component within the scope of the present disclosure.

When it is mentioned that one component is "connected" or "accessed" to another component, it may be understood that the one component is directly connected or accessed to another component or that still other component is interposed between the two components.

The singular forms "a", "an", and "the" are intended to include the plural forms as well, unless the context clearly indicates otherwise. As used herein, "A or B", "at least one of A and B", "at least one of A or B", "A, B or C", "at least one of A, B and C", and "at least one of A, B, or C," each of which may include any one of the items listed together in the corresponding one of the phrases, or all possible combinations thereof. It will be further understood that the terms "comprises" and/or "comprising," when used in this specification, specify the presence of stated features, integers, steps, operations, elements, components or a combination thereof, but do not preclude the presence or addition of one or more other features, integers, steps, operations, elements, components, and/or groups thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

As used in connection with the present disclosure, the term "module" may include a unit implemented in hardware, software, or firmware, and may interchangeably be used with other terms, for example, "logic," "logic block," "part," or "circuitry". A module may be a single integral component, or a minimum unit or part thereof, adapted to perform one or more functions. For example, according to an embodiment, the module may be implemented in a form of an application-specific integrated circuit (ASIC).

The term "unit" used herein may refer to a software or hardware component, such as a field-programmable gate array (FPGA) or an ASIC, and the "unit" performs predefined functions. However, "unit" is not limited to software or hardware. The "unit" may be configured to reside on an addressable storage medium or configured to operate one or more processors. Accordingly, the "unit" may include, for example, components, such as software components, object-oriented software components, class components, and task components, processes, functions, attributes, procedures, sub-routines, segments of program code, drivers, firmware, microcode, circuitry, data, databases, data structures, tables, arrays, and variables. The functionalities provided in the components and "units" may be combined into fewer components and "units" or may be further separated into additional components and "units." Furthermore, the components and "units" may be implemented to operate on one or more central processing units (CPUs) within a device or a security multimedia card. In addition, "unit" may include one or more processors.

Hereinafter, embodiments will be described in detail with reference to the accompanying drawings. When describing the embodiments with reference to the accompanying drawings, like reference numerals refer to like components and a repeated description related thereto will be omitted.

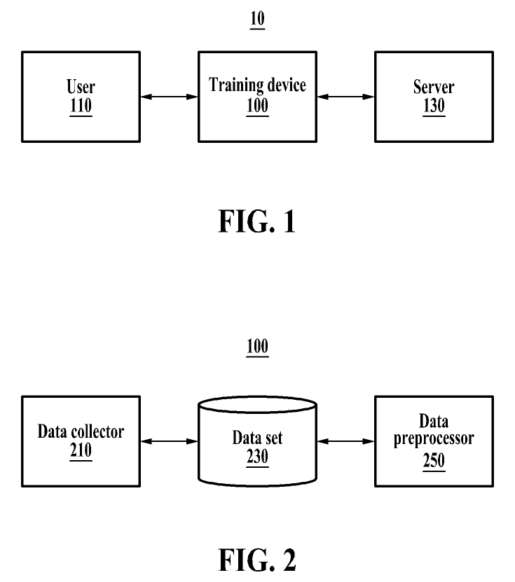

FIG. 1 is a schematic diagram illustrating a system of training an emotional situation recognition model, according to an embodiment.

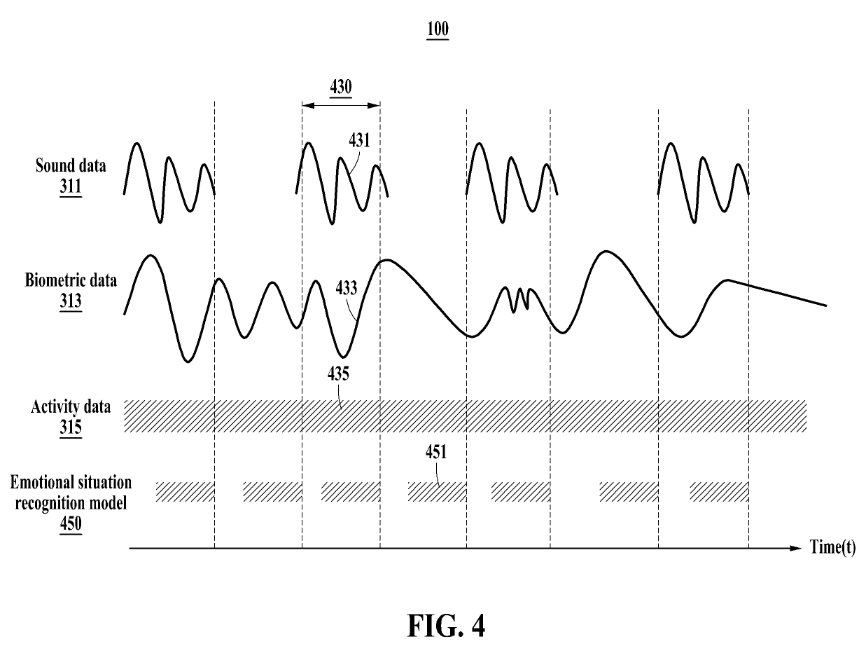

Referring to FIG. 1, according to an embodiment, a system 10 of training an emotional situation recognition model may include a training device 100, a user 110, and/or a server 130. The training device 100 may be a device for training an emotional situation recognition model (e.g., an emotional situation recognition model 450 of FIG. 4) for recognizing emotions of the user 110. The training device 100 may obtain data about the user 110. For example, the training device 100 may obtain profile information of the user 110, lifelog data of the user 110, and/or emotion journal data written about a first emotion caused by an event. The training device 100 may obtain data about each user 110 in different methods and may obtain the data at different times or simultaneously.

The training device 100 may transmit and upload the obtained user 110 data to the server 130. Based on information obtained through the server 130, the training device 100 may train the emotional situation recognition model 450.

FIG. 2 is a diagram illustrating a training device disclosed in FIG. 1.

Referring to FIG. 2, according to an embodiment, the training device 100 may include a data collector 210 and/or a data preprocessor 250. The data collector 210 and/or the data preprocessor 250, which are included in the training device 100 and perform respective functions, may vary in form depending on the type or environment of a device used. The data collector 210 may obtain information about a user (e.g., the user 110 of FIG. 1). For example, the data collector 210 may obtain profile information of the user 110, lifelog data of the user 110, and/or emotion journal data written about a first emotion caused by an event. The profile information may include the gender, age, and/or personality of the user 110. The profile information may further include information about mental health of the user 110. The lifelog data may include a plurality of types of lifelog data, and the plurality of types of lifelog data may include sound data of surroundings of the user 110, biometric data of the user 110, and/or activity data of the user 110. The plurality of types of lifelog data may further include image data and/or video data. The plurality of types of lifelog data is not limited to these types and may also include data generated based on the plurality of types of lifelog data. For example, the plurality of types of lifelog data may further include circumstantial data of the user 110 determined based on the sound data of the user 110 and the activity data of the user 110. When the activity data includes location data (or GPS data) about a location visited by the user 110, the circumstantial data may include information about a situation expected to occur at the location visited by the user 110. For example, when the location visited by the user 110 is a swimming pool, the circumstantial data may include information about a situation in which the user 110 is swimming. The sound data may include voices generated from a conversation partner of the user 110 and/or meeting.

The biometric data may include a heart rate of the user 110, heart rate variation (HRV), galvanic skin response (GSR), and/or photoplethysmogram (PPG). The biometric data may relate to normal or abnormal state information about the body of the user 110.

The emotion journal data generated about the first emotion caused by the event may include an utterance of the user 110 and text input by the user 110. The emotion journal data may be a self-report of the first emotion of the user 110.

The data collector 210 may transfer (or transmit) the obtained plurality of types of data to a data set 230. The data set 230 may be located within a training device (e.g., the training device 100 of FIG. 1). The data collector 210 may also store the obtained plurality of types of data in a server (e.g., the server 130 of FIG. 1) connected via a network. The data preprocessor 250 may generate data for training an emotional situation recognition model (e.g., the emotional situation recognition model 450 of FIG. 4).

The data preprocessor 250 may preprocess the plurality of types of data stored in the data set 230 to train the emotional situation recognition model 450. For example, the data preprocessor 250 may extract context information about the first emotion based on the emotion journal data. The context information may include the event, a time interval in which the event occurred, the first emotion, and/or location information in which the event occurred.

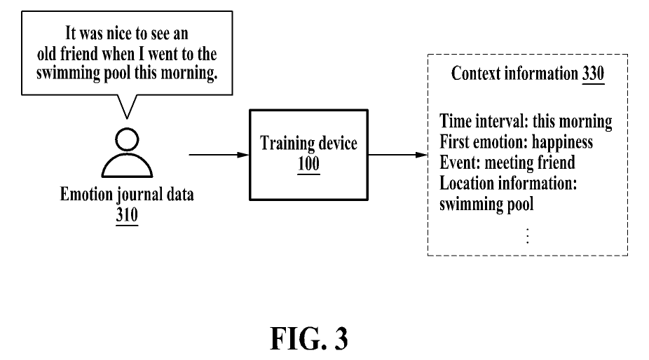

The data preprocessor 250 may segment only lifelog data corresponding to the time interval in which the event occurred from each piece of lifelog data based on the context information. The data preprocessor 250 may label each piece of the segmented lifelog data with the first emotion. The operation of preprocessing the lifelog data is described in detail with reference to FIGS. 3 to 5.

FIG. 3 is a diagram illustrating a method of obtaining context information, according to an embodiment.

Referring to FIG. 3, according to an embodiment, the training device 100 may obtain emotion journal data 310. The emotion journal data 310 may be a self-report of the user 110 about a first emotion. The training device 100 may obtain an utterance of the user 110. For example, the training device 100 may obtain the utterance from the user 110 such as "It was nice to see an old friend when I went to the swimming pool this morning".

The training device 100 may extract context information 330 about the first emotion based on the emotion journal data 310. The training device 100 may input the obtained utterance of the user 110 to a large language model (LLM) (not shown). The LLM may be a model installed on a server (e.g., the server 130 of FIG. 1) connected to the training device 100 via a network (not shown). For example, the network may include a local area network (LAN), a wide-area network (WAN), a value-added network (VAN), a mobile radio communication network, a satellite communication network, and a combination thereof. The network may be a comprehensive data communication network that allows the training device 100 and the server 130 to communicate smoothly with each other and may include wired Internet, wireless Internet, and a mobile wireless communication network. In addition, the wireless communication network may include, but is not limited to, wireless LAN (e.g., wireless fidelity (Wi-Fi)), Bluetooth, Bluetooth low energy, Zigbee, Wi-Fi Direct (WFD), ultra-wideband (UWB), infrared communication (e.g., infrared data association (IrDA)), near field communication (NFC), or the like.

The training device 100 may input the obtained utterance (e.g., "It was nice to see an old friend when I went to the swimming pool this morning") of the user 110 to the LLM to extract the context information 330 about the first emotion. For example, the training device 100 may input the utterance (e.g., "It was nice to see an old friend when I went to the swimming pool this morning") of the user 110 to the LLM to extract the first emotion (e.g., happiness) caused by an event (e.g., meeting a friend), the event, location information (e.g., the swimming pool) in which the event occurred, and/or a time interval (e.g., this morning) in which the event occurred. When the obtained utterance of the user 110 does not include the first emotion, the training device 100 may input the utterance of the user 110 to the LLM to predict (or estimate) the first emotion caused by the event of the user 110. The training device 100 may obtain a label corresponding to the first emotion to segment lifelog data. The training device 100 may utilize an external model (e.g., the LLM) of the training device 100 to obtain the label corresponding to the first emotion.

The training device 100 may interoperate with a model trained based on information about mental health. The training device 100 may input the emotion journal data 310 to the model trained based on the information about mental health, thereby additionally generating (or obtaining) information about mental health of the user 110 including depression level and/or anxiety level of the user 110.

FIG. 4 is a diagram illustrating an operation of segmenting lifelog data, according to an embodiment.

Referring to FIG. 4, according to an embodiment, the training device 100 may obtain profile information. The profile information may include a propensity of a user (e.g., the user 110 of FIG. 1). The training device 100 may also obtain the propensity of the user 110 statistically from obtained lifelog data or through a machine training model external to the training device 100.

The propensity of the user 110 may differ in biometric data 313 and/or activity data 315 depending on whether a user group is high or low in neuroticism or extraversion. For example, a user group with high neuroticism and low extraversion may exhibit a propensity of, compared to other groups, lower intensity of activity (or behavior) or more frequent reporting of negative emotions in activity patterns that deviate from daily routines. As such, the propensity of the user 110 may influence each of the biometric data 313 and/or the activity data 315. Furthermore, during periods of high level of depression, the frequency of activities, visited places, and social relationships may decrease significantly. The training device 100 may obtain the propensity and/or mental health information of the user 110 based on the lifelog data of one or more users other than the user 110.

The training device 100 may periodically or aperiodically request emotion journal data from the user 110. For example, the training device 100 may request the emotion journal data in response to occurrence of an event. The user 110 may periodically or aperiodically input the emotion journal data in response to the request for the emotion journal data from the training device 100. The training device 100 may obtain the emotion journal data through a user interface (UI). For example, the training device 100 may obtain the emotion journal data based on a click related to an emotion of the user 110, an utterance of the user 110, and/or a text input by the user 110.

The training device 100 may obtain a time interval 430 (e.g., this morning) in which the event occurred. The training device 100 may obtain information about a start point and an end point of the time interval 430. The training device 100 may set the start point and the end point of the event to obtain the time interval 430. The training device 100 may segment lifelog data corresponding to the time interval 430 in which the event occurred.

The training device 100 may segment sound data 431 corresponding to the time interval 430 from sound data 311. For example, the training device 100 may segment the sound data 431 corresponding to a time interval in which voice occurred in a swimming pool. The training device 100 may segment biometric data 433 corresponding to the time interval 430 from biometric data 313. In another example, the training device 100 may segment activity data 435 corresponding to the time interval 430 from the activity data 315.

The method of setting the time interval 430 or obtaining a first emotion may vary depending on the method of obtaining the emotion journal data (e.g., the emotion journal data 310 of FIG. 3) of the user 110. For example, when the emotion journal data 310 is obtained simply through a click from the user 110, the training device 100 may map a clicked emotion to the first emotion. In another example, when the emotion journal data 310 is obtained through the utterance of the user 110 and/or the text input by the user 110, the training device 100 may input the emotion journal data 310 to an LLM to extract context information (e.g., the context information 330 of FIG. 3) and may map an emotion included in the context information to the first emotion.

The training device 100 may label each piece of the segmented lifelog data (e.g., the sound data 431, the biometric data 433, and the activity data 435) with the first emotion (e.g., happiness). The training device 100 may store the lifelog data labeled with the first emotion (e.g., happiness) in a data set (e.g., the data set 230 of FIG. 2). The training device 100 may segment the lifelog data labeled with the first emotion into one or more segments and may use the lifelog data as training data for training the emotional situation recognition model 450.

The training device 100 may cause the emotional situation recognition model 450 to recognize a second emotion based on the lifelog data labeled with the first emotion (e.g., happiness). The emotional situation recognition model 450 may be a pre-trained general model, not a model for recognizing emotions of the user 110. The lifelog data labeled with the first emotion (e.g., happiness) may be a retraining data set for retraining the emotional situation recognition model 450, which is a pre-trained general-purpose model. The training device 100 may generate the lifelog data labeled with the first emotion (e.g., happiness) as an additional column for a plurality of types of lifelog data (e.g., the sound data 311, the biometric data 313, and the activity data 315) or may store the lifelog data separately in the data set 230.

The emotional situation recognition model 450 may recognize the second emotion at a predetermined interval. For example, the emotional situation recognition model 450 may recognize the second emotion in a section 451. The emotional situation recognition model 450 may recognize the second emotion based on data obtained up to a point in time in which the second emotion is recognized. As a data set trained by the emotional situation recognition model 450 changes, the second emotion recognized may change. For example, the emotional situation recognition model 450 may recognize the second emotion as more similar to the first emotion extracted from the utterance of the user 110 over time.

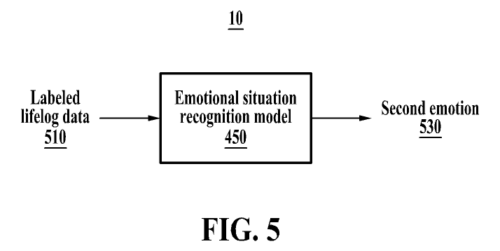

FIG. 5 is a diagram illustrating a method of training an emotional situation recognition model, according to an embodiment.

Referring to FIG. 5, according to an embodiment, a training device (e.g., the training device 100 of FIG. 1) may train the emotional situation recognition model 450. The training device 100 may label each piece of segmented lifelog data (e.g., the sound data 431, the biometric data 433, and the activity data 435) to generate labeled lifelog data 510.

The training device 100 may train the emotional situation recognition model 450 to recognize a second emotion 530 of a user (e.g., the user 110 of FIG. 1) based on profile information and the labeled lifelog data 510. The training device 100 may periodically determine a point in time for training the emotional situation recognition model 450 based on the profile information and the labeled lifelog data 510. The training device 100 may also determine the point in time for training the emotional situation recognition model 450 based on a policy. For example, the training device 100 may train the emotional situation recognition model 450 based on a disparity between an emotion recognition result of a pre-trained emotional situation recognition model based on a preset amount of lifelog data and the second emotion 530 recognized by the emotional situation recognition model 450. Here, the policy may be a criterion for determining the disparity between the emotion recognition result of the pre-trained emotional situation recognition model based on the preset amount of lifelog data and the second emotion 530 recognized by the emotional situation recognition model 450.

The training device 100 may cause the emotional situation recognition model 450 to provide the second emotion 530. The training device 100 may obtain a satisfaction level for the emotional situation recognition model 450 that provides the second emotion 530. For example, the training device 100 may receive the satisfaction level for the emotional situation recognition model 450 from the user 110 through a UI of the emotional situation recognition model 450. The training device 100 may train the emotional situation recognition model 450 based on the satisfaction level for the emotional situation recognition model 450.

The training device 100 may train the emotional situation recognition model 450 based on a retraining data set, and the emotional situation recognition model 450 may be enhanced into a personalized model. The training device 100 may train the emotional situation recognition model 450 through an actor-critic algorithm. For example, the training device 100 may cause the emotional situation recognition model 450 to recognize the second emotion 530 and may train the emotional situation recognition model 450 based on feedback (or reward) about accuracy of the recognition. The training device 100 may cause the emotional situation recognition model 450 to recognize the second emotion 530 and may train the emotional situation recognition model 450 based on a difference between a first emotion and the second emotion 530. The training device 100 may use the actor-critic algorithm to train the emotional situation recognition model 450 in patterns of emotions of the user 110. The reward for the accuracy of the recognition may be set based on emotion journal data.

The training device 100 may obtain each piece of lifelog data of one or more users other than the user 110. Based on each piece of the lifelog data of one or more users, the training device 100 may train one or more emotional situation recognition models (e.g., the emotional situation recognition model 450) using a meta-learning method. The training device 100 may fine-tune the emotional situation recognition models trained using the meta-learning method to generate a personalized model for the user 110.

The second emotion 530 recognized by the emotional situation recognition model 450 may be based on the first emotion included in context information, an arousal level representing a degree of emotional arousal, emotional valence, and/or emotion score. For example, when the user 110 is excited and shouts, the emotional situation recognition model 450 may recognize a high arousal level and based on this high arousal level, may recognize the second emotion 530. In another example, when laughter of the user 110 is obtained as sound data, the emotional situation recognition model 450 may assign a positive value to the emotional valence and based on this positive emotional valence, may recognize the second emotion 530.

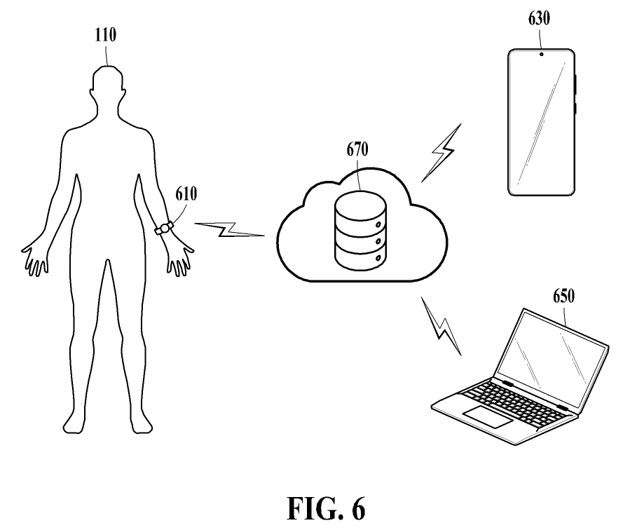

FIG. 6 is a diagram illustrating a method of obtaining lifelog data and emotion journal data, according to an embodiment.

Referring to FIG. 6, according to an embodiment, a training device (e.g., the training device 100 of FIG. 1) may obtain lifelog data and/or emotion journal data through various routes.

The system 10 of training an emotional situation recognition model may include a device 610 worn by or attached to the user 110, a terminal device 630 of the user 110, and an electronic device 650 with which the user 110 may communicate. For example, the device 610 may include a wearable device and/or a smart watch. The terminal device 630 may include a smartphone, a tablet personal computer (PC), and/or a mobile phone. The electronic device 650 may include, but is not limited to, a speaker (e.g., an artificial intelligence (AI) speaker), a video phone, a desktop PC, a laptop PC, and/or a netbook computer.

The device 610 may obtain location information of the user 110 and/or weather information of a location in which the user 110 is located. The device 610 may further obtain sound information about surroundings of the user 110. The device 610 may obtain a self-report about a first emotion from the user 110 through an application installed on the device 610.

The training device 100 may store the lifelog data and/or the emotion journal data obtained through various routes in a server 670. The server 670 may be implemented externally to the training device 100.

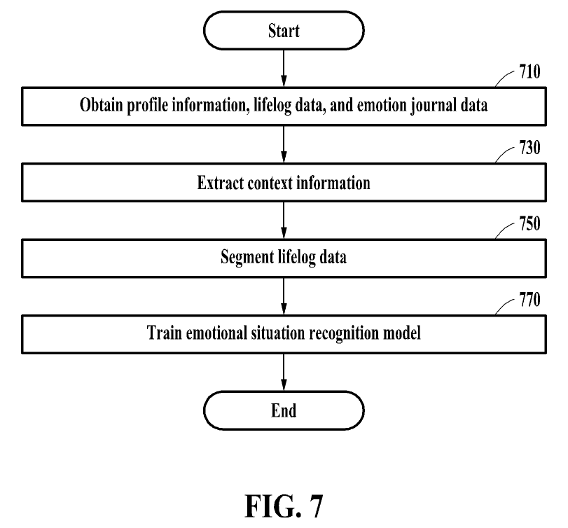

FIG. 7 is a flowchart illustrating a method of training an emotional situation recognition model, according to an embodiment.

Referring to FIG. 7, according to an embodiment, operations 710 to 770 may be operations performed by the training device 100 of FIG. 1 described with reference to FIGS. 1 to 8.

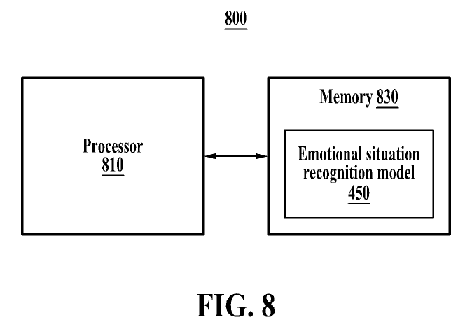

According to an embodiment, operations 710 to 770 may be understood to be performed by the training device 100 (e.g., an electronic device 800 of FIG. 8) described with reference to FIG. 1.

In operation 710, the training device 100 may obtain profile information of a user (e.g., the user 110 of FIG. 1), lifelog data of the user 110, and emotion journal data (e.g., the emotion journal data 310 of FIG. 3) written about a first emotion caused by an event. The emotion journal data 310 may include an utterance of the user 110 and a text input by the user 110.

In operation 730, the training device 100 may extract context information (e.g., the context information 330 of FIG. 3) about the first emotion based on the emotion journal data 310. The context information 330 may include information about the event, a time interval in which the event occurred (e.g., the time interval 430 of FIG. 4), the first emotion, and a location in which the event occurred.

In operation 750, the training device 100 may segment only lifelog data corresponding to the time interval 430 in which the event occurred from each piece of the lifelog data based on the context information 330. The lifelog data may include a plurality of types of lifelog data, and the plurality of types of lifelog data may include sound data (e.g., the sound data 311 of FIG. 4) of surroundings of the user 110, biometric data (e.g., the biometric data 313 of FIG. 4) of the user 110, and/or activity data (e.g., the activity data 315 of FIG. 4) of the user 110. The plurality of types of lifelog data may further include circumstantial data about a situation of the user 110 determined based on the activity data 315. The training device 100 may segment the circumstantial data corresponding to activity data (e.g., the activity data 435 of FIG. 4).

In operation 770, the training device 100 may train an emotional context recognition model (e.g., the emotional context recognition model 450 of FIG. 4) for recognizing a second emotion (e.g., the second emotion 530 of FIG. 5) of the user 110 based on the profile information, the segmented lifelog data, and the context information 330. The training device 100 may train the emotional context recognition model 450 based on the context information 330 and the segmented lifelog data. For example, the training device 100 may train the emotional context recognition model 450 based on the activity data 435 and the circumstantial data in the same time interval 430 as the context information 330.

Operations 710 to 770 may be performed sequentially but are not limited thereto. For example, two or more operations may be performed in parallel.

FIG. 8 is a schematic block diagram of a training device according to an embodiment.

Referring to FIG. 8, according to an embodiment, an electronic device 800 (e.g., the training device 100 of FIG. 1) may include a memory 830 and a processor 810.

The memory 830 may store instructions (or programs) executable by the processor 810. For example, the instructions may include instructions for executing operations of the processor 810 and/or operations of each component of the processor 810.

The memory 830 may include the trained emotional situation recognition model 450. The memory 830 may store data required for the emotional situation recognition model 450 to recognize a second emotion (e.g., the second emotion 530 of FIG. 5).

The memory 830 may include one or more computer-readable storage media. The memory 830 may include non-volatile storage devices (e.g., a magnetic hard disk, an optical disk, a floppy disk, flash memory, electrically programmable read-only memory (EPROM), or electrically erasable programmable read-only memory (EEPROM).

The memory 830 may be a non-transitory medium. The term "non-transitory" may represent that the storage medium is not implemented as a carrier wave or a propagated signal. However, the term "non-transitory" should not be construed as meaning that the memory 830 is immovable.

The processor 810 may process the data stored in the memory 830. The processor 810 may execute computer-readable code (e.g., software) stored in the memory 830 and the instructions generated by the processor 810.

The processor 810 may be a data processing device implemented by hardware including a circuit having a physical structure to perform desired operations. For example, the desired operations may include code or instructions included in programs.

The hardware-implemented data processing device may include, for example, a microprocessor, a CPU, a processor core, a multi-core processor, a multiprocessor, an ASIC, and an FPGA.

The processor 810 may cause the electronic device 800 to perform one or more operations by executing the code and/or instructions stored in the memory 830. The operations performed by the electronic device 800 may be substantially the same as the operations performed by the training device 100 described with reference to FIGS. 1 to 8. Accordingly, any redundant description is omitted.

The components described in the embodiments may be implemented by hardware components including, for example, at least one digital signal processor (DSP), a processor, a controller, an ASIC, a programmable logic element, such as an FPGA, other electronic devices, or combinations thereof. At least some of the functions or the processes described in the embodiments may be implemented by software, and the software may be recorded on a recording medium. The components, the functions, and the processes described in the embodiments may be implemented by a combination of hardware and software.

The embodiments described herein may be implemented using hardware components, software components, or a combination thereof. A processing device may be implemented using one or more general-purpose or special purpose computers, such as, for example, a processor, a controller and an arithmetic logic unit, a DSP, a microcomputer, an FPGA, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such as parallel processors.

The software may include a computer program, a piece of code, an instruction, or some combination thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums.

The method according to the above-described embodiments may be recorded in non-transitory computer-readable media including program instructions to implement various operations which may be performed by a computer. The media may also include, alone or in combination with the program instructions, data files, data structures, and the like. The program instructions recorded on the media may be those specially designed and constructed for the purposes of the embodiments, or they may be of the well-known kind and available to those having skill in the computer software arts. Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as code produced by a compiler, and files containing higher level code that may be executed by the computer using an interpreter.

The described hardware devices may be configured to act as one or more software modules in order to perform the operations of the above-described embodiments, or vice versa.

While this disclosure includes embodiments, it will be apparent to one of ordinary skill in the art that various changes in form and details may be made in these embodiments without departing from the spirit and scope of the claims and their equivalents. For example, suitable results may be achieved if the described techniques are performed in a different order, and/or if components in a described system, architecture, device, or circuit are combined in a different manner and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. A method of training an emotional situation recognition model, the method comprising:
obtaining profile information of a user, lifelog data of the user, and emotion journal data written about a first emotion caused by an event;
extracting context information about the first emotion based on the emotion journal data;
segmenting only lifelog data corresponding to a time interval in which the event occurred from each piece of the lifelog data, based on the context information; and
training the emotional situation recognition model for recognizing a second emotion of the user, based on the profile information, the segmented lifelog data, and the context information.

2. The method of claim 1, wherein
the emotion journal data comprises an utterance of the user and a text input by the user.

3. The method of claim 1, wherein
the context information comprises the event, the time interval, the first emotion, and location information in which the event occurred.

4. The method of claim 1, wherein
the lifelog data comprises a plurality of types of lifelog data, and
the plurality of types of lifelog data comprises sound data of surroundings of the user, biometric data of the user, and activity data of the user.

5. The method of claim 1, wherein
the training comprises labeling each piece of the segmented lifelog data with the first emotion.

6. The method of claim 5, wherein
the training comprises recognizing the second emotion based on the lifelog data labeled with the first emotion.

7. The method of claim 6, wherein
the training comprises training the emotional situation recognition model based on the first emotion and the second emotion.

8. The method of claim 1, wherein
the extracting comprises inputting the emotion journal data to a large language model (LLM) to extract the context information about the first emotion.

9. The method of claim 1, wherein
the profile information comprises gender, age, and personality of the user.

10. A non-transitory computer-readable storage medium storing instructions that, when executed by a processor, cause the processor to perform the method of claim 1.

11. An electronic device comprising:
a memory comprising instructions; and
at least one processor electrically connected to the memory and configured to execute the instructions,
wherein the instructions, when executed by the at least one processor, cause the electronic device to:
obtain profile information of a user, lifelog data of the user, and emotion journal data written about a first emotion caused by an event;
extract context information about the first emotion based on the emotion journal data;
segment only lifelog data corresponding to a time interval in which the event occurred from each piece of the lifelog data, based on the context information; and
train an emotional situation recognition model for recognizing a second emotion of the user, based on the profile information, the segmented lifelog data, and the context information.

12. The electronic device of claim 11, wherein
the emotion journal data comprises an utterance of the user and a text input by the user.

13. The electronic device of claim 11, wherein
the context information comprises the event, the time interval, the first emotion, and location information in which the event occurred.

14. The electronic device of claim 11, wherein
the lifelog data comprises sound data of surroundings of the user, biometric data of the user, and activity data of the user.

15. The electronic device of claim 11, wherein
the instructions, when executed by the at least one processor, cause the electronic device to label each piece of the segmented lifelog data with the first emotion.

16. The electronic device of claim 15, wherein
the instructions, when executed by the at least one processor, cause the electronic device to recognize the second emotion based on the lifelog data labeled with the first emotion.

17. The electronic device of claim 16, wherein
the instructions, when executed by the at least one processor, cause the electronic device to train the emotional situation recognition model based on the first emotion and the second emotion.

18. The electronic device of claim 11, wherein
the instructions, when executed by the at least one processor, cause the electronic device to input the emotion journal data to a large language model (LLM) to extract the context information about the first emotion.

19. The electronic device of claim 11, wherein
the profile information comprises gender, age, and personality of the user.
